(19) United States
(12) Patent Application Publication
(10) Pub. No.: US 2026/0232495 A1
(43) Pub. Date: Aug. 13, 2026

(54) DRESSING DEVICE FOR NEGATIVE PRESSURE WOUND THERAPY SYSTEM

(71) Applicant: Solventum Intellectual Properties Company, Maplewood, MN (US)

(72) Inventors: Benjamin A. PRATT, Poole (GB); Thomas A. EDWARDS, Hampshire (GB); Timothy M. ROBINSON, Shillingstone (GB)

(21) Appl. No.: 19/156,761

(22) PCT Filed: Feb. 21, 2024
(86) PCT No.: PCT/IB2024/051674

§ 371 Date: Aug. 14, 2025

Related U.S. Application Data
(60) Provisional application No. 63/447,476, filed on Feb. 22, 2023.

(51) Int. Cl. A61F 13/05; A61F 13/00; A61F 13/0203; A61M 1/00

(52) U.S. Cl. CPC A61F 13/05; A61F 13/0226; A61M 1/915; A61F 2013/00604; A61F 2013/00778

(57) ABSTRACT

A dressing device for a negative pressure wound therapy includes a base film configured to couple to a wound site. The dressing device further includes a manifold coupled to the base film. The manifold includes a first portion configured to interface with the wound site and a second portion spaced apart from the wound site. The dressing device further includes an absorbent spaced apart from the first portion of the manifold. The absorbent is coupled to and extends along the second portion of the manifold, such that the absorbent is offset from the wound site. The dressing device further includes a top film disposed adjacent to the absorbent opposite to the manifold, such that the absorbent is disposed between the manifold and the top film.

### CROSS-REFERENCE TO RELATED APPLICATIONS

This application is a U.S. National Stage Entry of PCT International Application No. PCT/IB2024/051674, filed Feb. 21, 2024, which claims the benefit of priority to U.S. Provisional Application No. 63/447,476, filed on Feb. 22, 2023, which is incorporated herein by reference in its entirety.

### TECHNICAL FIELD

The present disclosure generally relates to a negative pressure wound therapy (NPWT) system and more particularly to a dressing device for use with the NPWT system.

### BACKGROUND

Caring for wounds is important in a healing process. Wounds generally produce fluids, commonly referred to as wound exudate. The wound exudate may include slough, necrotic tissue, or microbial load (e.g., bacteria and biofilms). If not properly addressed, the wound exudate at a wound site can lead to infection or maceration.

Negative pressure wound therapy (NPWT) systems are embodied as sealed wound-care systems particularly indicated for wounds, such as, chronic persistent wounds and/or complicated wounds. Specifically, for promoting wound healing, a pressure that is reduced relative to the surroundings (commonly referred to as “negative pressure”) is applied to the wound site. The negative pressure causes mechanical contraction of the wound and removal of the wound exudate from the wound site, thus promoting formation of granulation tissues and accelerating wound healing. The NPWT system typically includes a therapy unit that is in fluid communication with the wound site.

In some examples, conventional wound dressings or bridge dressings may include a superabsorbent to absorb low levels of the wound exudates. The superabsorbent is typically disposed at a wound interface such that the superabsorbent may directly communicate with the wound site. When such wound dressings or bridge dressings get saturated with the wound exudate, the wound dressings or bridge dressings may adversely affect a transfer of the negative pressure to the wound site as the wound exudate may block pathways for negative pressure flow, which may lead to loss of therapy. In other cases, the wound exudate collected in the wound dressing or bridge dressing itself may present a risk of skin maceration if the absorbent is fully saturated. Further, some wound dressings or bridge dressings may not completely isolate components of the NPWT system, such as, a pump from the wound exudate. Thus, some wound dressings or bridge dressings may allow ingress of the wound exudate into the components of the NPWT system, which may contaminate and/or damage the components of the NPWT system.

Typically, a dressing interface may facilitate coupling of the therapy unit to the wound dressing of the NPWT system. A low-profile dressing interface can improve patient comfort and reduce a risk of non-compliance. Currently, the low-profile dressing interface is supplied as a separate part to the wound dressing, which may increase a number of parts associated with the NPWT system.

### SUMMARY

Generally, the present disclosure relates to a dressing device and a negative pressure wound therapy (NPWT) system including the dressing device.

In a first aspect, the present disclosure provides a dressing device for a NPWT system. The dressing device includes a base film configured to couple to a wound site. The dressing device further includes a manifold coupled to the base film. The manifold includes a first portion configured to interface with the wound site and a second portion spaced apart from the wound site. The dressing device further includes an absorbent spaced apart from the first portion of the manifold. The absorbent is coupled to and extends along the second portion of the manifold, such that the absorbent is offset from the wound site. The dressing device further includes a top film disposed adjacent to the absorbent opposite to the manifold, such that the absorbent is disposed between the manifold and the top film.

In a second aspect, the present disclosure provides a NPWT system for a wound site. The NPWT system includes a negative pressure source configured to apply a negative pressure at the wound site. The NPWT system further includes a pressure sensing device configured to determine a pressure proximal to the wound site. The NPWT system further includes the dressing device of the first aspect. The dressing device fluidly communicates the negative pressure source with the wound site. The dressing device further fluidly communicates the pressure sensing device with the wound site.

In a third aspect, the present disclosure provides a dressing device for a NPWT system. The dressing device includes a base film configured to couple to a wound site. The dressing device further includes a manifold coupled to the base film. The manifold includes a first portion configured to interface with the wound site and a second portion spaced apart from the wound site. The manifold further includes a manifold channel extending along the manifold and fluidly communicating with the wound site. The dressing device further includes a top film disposed adjacent to the manifold opposite to the base film, such that the manifold is disposed between the base film and the top film. The top film includes a top channel extending along the top film and aligned with the manifold channel. The manifold channel and the top channel together define a pressure sensing pathway therebetween.

In a fourth aspect, the present disclosure provides a NPWT system for a wound site. The NPWT system includes a negative pressure source configured to apply a negative pressure at the wound site. The NPWT system further includes a pressure sensing device configured to determine a pressure proximal to the wound site. The NPWT system further includes the dressing device of the third aspect. The dressing device fluidly communicates the negative pressure source with the wound site. The dressing device further fluidly communicates the pressure sensing device with the wound site.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 is a schematic view of a negative pressure wound therapy (NPWT) system for a wound site, according to an embodiment of the present disclosure;

FIG. 2A is a schematic perspective view of a dressing device associated with the NPWT system of FIG. 1, according to an embodiment of the present disclosure;

FIG. 2B is a schematic exploded perspective view of the dressing device of FIG. 2A;

FIG. 2C is a schematic partial sectional side view of the dressing device of FIG. 2A;

FIG. 3A is a schematic top view of a manifold of the dressing device of FIG. 2A;

FIG. 3B is a schematic side perspective view of the manifold of the dressing device of FIG. 2A;

FIG. 3C is a schematic top view of an absorbent of the dressing device of FIG. 2A;

FIG. 4A is a schematic perspective view of a dressing device that may be associated with the NPWT system of FIG. 1, according to another embodiment of the present disclosure;

FIG. 4B is a schematic partial sectional side view of the dressing device of FIG. 4A;

FIG. 5A is a schematic exploded perspective view of a dressing device that may be associated with the NPWT system of FIG. 1, according to another embodiment of the present disclosure;

FIG. 5B is a schematic partial sectional side view of the dressing device of FIG. 5A;

FIG. 6A is a schematic exploded perspective view of a dressing device that may be associated with the NPWT system of FIG. 1, according to another embodiment of the present disclosure;

FIG. 6B is a schematic partial sectional side view of the dressing device of FIG. 6A;

FIG. 7A is a schematic exploded perspective view of a dressing device that may be associated with the NPWT system of FIG. 1, according to another embodiment of the present disclosure;

FIG. 7B is a schematic partial sectional side view of the dressing device of FIG. 7A;

FIG. 8 is a schematic exploded perspective view of a dressing device that may be associated with the NPWT system of FIG. 1, according to another embodiment of the present disclosure;

FIG. 9A is a schematic exploded perspective view of a dressing device that may be associated with the NPWT system of FIG. 1, according to another embodiment of the present disclosure;

FIG. 9B is a schematic top view of the dressing device of FIG. 9A;

FIG. 9C is a magnified schematic sectional perspective view of a portion A of the dressing device of FIG. 9B;

FIG. 9D is a magnified schematic sectional perspective view of a portion B of the dressing device of FIG. 9B;

FIG. 9E is a magnified schematic sectional side view of a portion C of the dressing device of FIG. 9B;

FIG. 10A is a schematic exploded perspective view of a dressing device that may be associated with the NPWT system of FIG. 1, according to another embodiment of the present disclosure;

FIG. 10B is a schematic top view of the dressing device of FIG. 10A;

FIG. 10C is a magnified schematic exploded sectional side view of a portion D of the dressing device of FIG. 10B; and

FIG. 10D is a magnified schematic sectional perspective view of a portion E of the dressing device of FIG. 10B.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As used herein, all numbers should be considered modified by the term “about”. As used herein, “a,” “an,” “the,” “at least one,” and “one or more” are used interchangeably.

The term “about”, unless otherwise specifically defined, means to a high degree of approximation (e.g., within +/-5% for quantifiable properties) but again without requiring absolute precision or a perfect match.

As used herein as a modifier to a property or attribute, the term “generally”, unless otherwise specifically defined, means that the property or attribute would be readily recognizable by a person of ordinary skill but without requiring absolute precision or a perfect match (e.g., within +/-20 % for quantifiable properties).

The term “substantially”, unless otherwise specifically defined, means to a high degree of approximation (e.g., within +/-10% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, “at least one of A and B” should be understood to mean “only A, only B, or both A and B”.

The term “coupled”, or “connected” may include direct physical connections between two or more components, or indirect physical connections between two or more components that are connected together by one or more additional components. For example, a first component may be coupled to a second component by being directly connected together or by being connected by a third component.

As used herein, the term “configured to” and like is at least as restrictive as the term “adapted to” and requires actual design intention to perform the specified function rather than mere physical capability of performing such a function.

As used herein, the terms “layer,” “sheet,” and “dressing,” or variations thereof, are used to describe an article having a thickness that is small relative to its length and width.

As used herein, the term “negative pressure” broadly refers to a pressure lower than a local pressure in a local environment outside of a sealed treatment environment provided by a dressing. In many cases, the local ambient pressure can also be the atmospheric pressure at which a wound site is located. Alternatively, the pressure can be less than a hydrostatic pressure associated with a tissue at the wound site.

As used herein, the term “wounds” may include, for example, chronic, acute, traumatic, subacute, closed surgical wounds or dehiscence wounds, partially thick burns, ulcers (such as, diabetic, compressive, or venous insufficiency ulcers), flaps, and grafts. The wound may also include an open abdomen area of a patient.

As used herein, the term “wound site” may include a tissue site, such as, bone tissue, adipose tissue, muscle tissue, nerve tissue, skin tissue, vascular tissue, connective tissue, cartilage, tendons, or ligaments. The term “wound site” may also refer to an area of a tissue that is not necessarily a wound or a defect but may be desired to add or promote additional tissue growth. For example, negative pressure therapy can be used in a particular tissue area to grow additional tissue that can be harvested or transplanted to another tissue site. The wound site may also include an area wherein a surgical incision has been previously performed.

As used herein, the term “transparent” refers to constructions that appear to be transparent to the naked human eye. The constructions may exhibit high light transmittance over at least a portion of the visible light spectrum (about 400 to about 700 nanometers), and low haze.

As used herein, the term “fluid permeable” may mean that a material or a component may allow passage of fluids, including liquids and gases, therethrough.

Negative pressure wound therapy (NPWT) systems are often used to promote wound healing. In order to heal a wound, a negative pressure is applied at a wound site via the NPWT system. Since the NPWT system fluidly communicates with the wound site, the NPWT system removes a fluid, i.e., a wound exudate from the wound site by applying the negative pressure on a wound dressing attached to the wound site. The wound exudate may include slough, necrotic tissue, microbial load (e.g., bacteria and biofilms), and the like. Further, the wound exudate is collected in a canister for disposal or analysis. The canister of the NPWT system generally includes a rigid canister. Typically, to manage low levels of the wound exudate, the use of rigid canisters may increase patient discomfort as they may make the NPWT system bulky to handle and may affect a mobility of patient. Further, such rigid canisters may impact a portability of the NPWT system. Thus, rigid canisters may be bulky, may confine a patient to bed or at least render the patient immobile, and may lead to patient discomfort.

In some examples, conventional wound dressings or bridge dressings may include a superabsorbent to absorb low levels of the wound exudate. The superabsorbent is typically disposed at a wound interface such that the superabsorbent may directly communicate with the wound site. When such wound dressings or bridge dressings get saturated with the wound exudate, the wound dressings or bridge dressings may adversely affect a transfer of the negative pressure to the wound site as the wound exudate may block pathways for negative pressure flow, which may lead to loss of therapy. In other cases, the wound exudate collected in the wound dressing or bridge dressing itself may present a risk of skin maceration if the absorbent is fully saturated. Further, some wound dressings or bridge dressings may not completely isolate components of the NPWT system, such as, a pump from the wound exudate. Thus, some wound dressings or bridge dressings may allow ingress of the wound exudate into the components of the NPWT system, which is not desirable.

Further, a dressing interface may facilitate coupling the therapy unit to the wound dressing of the NPWT system. A low-profile dressing interface can improve patient comfort and reduce the risk of non-compliance. Currently, the low-profile dressing interface is supplied as a separate part to the wound dressing, which may increase a number of parts associated with the NPWT system.

Therefore, there exists a need for a dressing device that, when used with an NPWT system, may allow collection of the wound exudate from the wound without disrupting negative pressure therapy at the wound site, without causing skin maceration, and without impacting patient comfort, while improving an ease of use, a portability, and an efficacy of the NPWT system.

The present disclosure relates to a dressing device for a NPWT system. The dressing device includes a base film configured to couple to a wound site. The dressing device further includes a manifold coupled to the base film. The manifold includes a first portion configured to interface with the wound site and a second portion spaced apart from the wound site. The dressing device further includes an absorbent spaced apart from the first portion of the manifold. The absorbent is coupled to and extends along the second portion of the manifold, such that the absorbent is offset from the wound site. The dressing device further includes a top film disposed adjacent to the absorbent opposite to the manifold, such that the absorbent is disposed between the manifold and the top film.

The offset between the absorbent and the wound site may prevent a direct contact between the wound exudate absorbed by the absorbent and the wound site. Since the absorbent is offset from the wound site, a possibility of skin maceration, when the absorbent is saturated with the wound exudate, may be reduced.

Further, in some embodiments, the first portion and/or the second portion of the manifold may include projections thereon. In such embodiments, the projections may define a negative pressure pathway even when the absorbent is saturated with the wound exudate. Thus, the dressing device may allow effective management of the wound exudate in all orientations and may reduce a possibility of blocking of negative pressure pathways when the absorbent is saturated with the wound exudate.

Overall, the dressing device as described herein may be easy to use, may improve patient comfort, may reduce a possibility of infection at the wound site, may improve an efficacy and a portability of the NPWT system, and may effectively manage the wound exudate removed from the wound site.

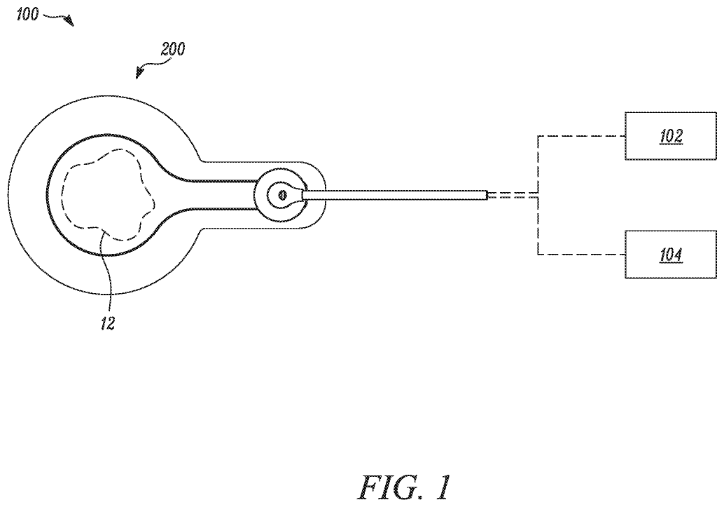

FIG. 1 is a schematic view illustrating a NPWT system 100 for a wound site 12, according to an embodiment of the present disclosure. The NPWT system 100 may be disposed on a skin (not shown) of a user (not shown) for treatment of the wound site 12. The user may be a patient having a wound (not shown). Wounds generally produce fluids, commonly referred to as wound exudate. The wound exudate may include slough, necrotic tissue, or microbial load (e.g., bacteria and biofilms). If not properly addressed, the wound exudate at the wound site 12 can lead to infection or maceration at the wound site 12.

The NPWT system 100 includes a negative pressure source 102 configured to apply a negative pressure at the wound site 12. The negative pressure causes mechanical contraction of the wound and removal of the wound exudate from the wound site 12, thus promoting formation of granulation tissues and accelerating wound healing. In some examples, the negative pressure source 102 may include a reservoir of air at a negative pressure or a manual or electrically powered device, such as, a pump. The pump may include, for example, a vacuum pump, a suction pump, or a micro-pump.

The NPWT system 100 further includes a pressure sensing device 104 configured to determine a pressure proximal to the wound site 12. The pressure sensing device 104 may be configured to provide information to a person, who can then manually control one or more operating parameters of the NPWT system 100 or control/replace one or more devices of the NPWT system 100 that may interact with the wound site 12. In some embodiments, the pressure sensing device 104 may include a piezoresistive strain gauge, without any limitations.

The negative pressure source 102 and the pressure sensing device 104 may form a part of a therapy unit (not shown) that may additionally include components, such as, a controller for controlling a negative pressure therapy being applied by the NPWT system 100, a battery pack, and the like. In some cases, the controller may automatically control the negative pressure applied to the wound site 12 based on signals received from the pressure sensing device 104. The battery pack may provide power to one or more components of NPWT system 100, such as, the negative pressure source 102 and/or the pressure sensing device 104. In some examples, the battery pack may include one or more batteries including, but not limited to, a nickel cadmium battery, a nickel metal hydride battery, a lithium-ion battery, a small-sealed lead acid battery, an alkaline battery, or any other suitable type of battery. The battery pack may be removable, may be wirelessly chargeable, and the like.

The NPWT system 100 further includes a dressing device 200. The dressing device 200 is configured to be disposed at the wound site 12. The dressing device 200 may at least partially face the wound site 12. The dressing device 200 is configured to be in fluid communication with the wound site 12. Specifically, the dressing device 200 fluidly communicates the negative pressure source 102 with the wound site 12. The dressing device 200 further fluidly communicates the pressure sensing device 104 with the wound site 12.

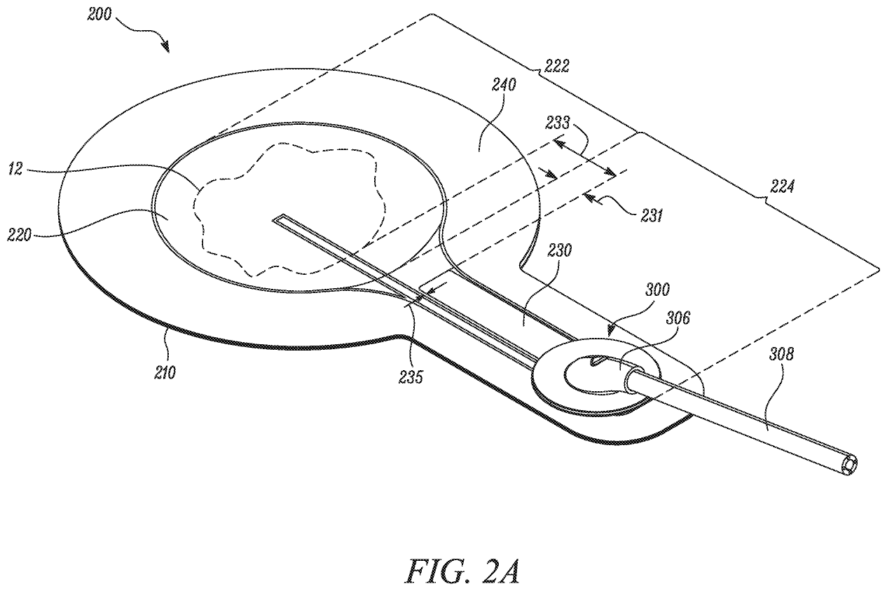

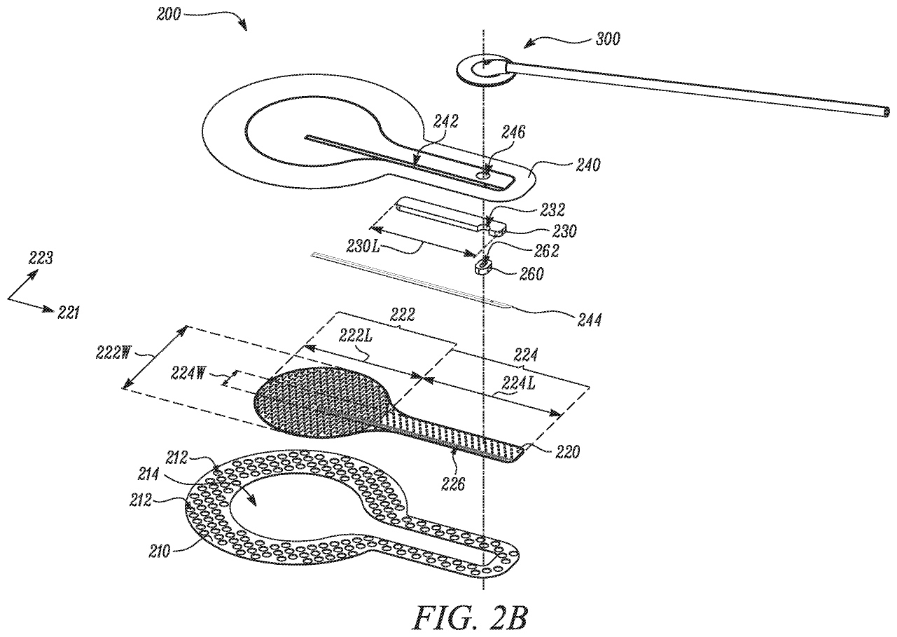

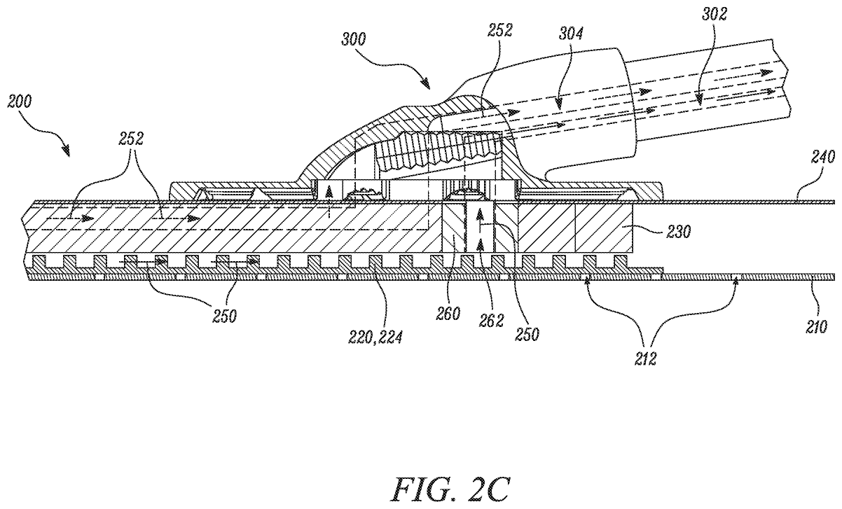

FIGS. 2A, 2B, and 2C illustrate the dressing device 200 for the NPWT system 100 of FIG. 1, according to an embodiment of the present disclosure. Specifically, FIG. 2A illustrates a schematic perspective view of the dressing device 200. FIG. 2B illustrates a schematic exploded perspective view of the dressing device 200. FIG. 2C illustrates a schematic partial sectional side view of the dressing device 200.

Referring to FIGS. 2A, 2B, and 2C, the dressing device 200 includes a base film 210, a manifold 220, an absorbent 230, and a top film 240.

The base film 210 is configured to couple to the wound site 12. In some embodiments, the base film 210 includes a plurality of perforations 212. The perforations 212 are circular in shape. However, the perforations 212 may include any other shape. In some embodiments, the base film 210 may be made of a silicone material.

Further, the manifold 220 may include any substance or structure providing a plurality of pathways adapted to collect fluid (e.g., the wound exudate) from the wound site 12 or distribute fluid (e.g., the negative pressure) across the wound site 12. For example, the manifold 220 may be adapted to receive the negative pressure from the negative pressure source 102 (see FIG. 1) and distribute the negative pressure across the wound site 12, which may have the effect of collecting the wound exudate from across the wound site 12 and drawing the wound exudate toward the negative pressure source 102.

The manifold 220 is coupled to the base film 210. In some embodiments, the base film 210 and the manifold 220 may be at least partially connected to each other at a periphery of the manifold 220. In some embodiments, the base film 210 may connect with the manifold 220 via a heat seal, a weld seam, adhesives, and the like. In some embodiments, the manifold 220 includes a transparent material. Therefore, the wound site 12 may be viewed without removing the dressing device 200. In some embodiments, the manifold 220 may be formed from Libeltex TDL2 having a material weight of 80 grams per square member (gsm). In other embodiments, the manifold 220 may have a material weight between about 20 gsm and about 140 gsm. Larger material weights may be selected to increase manifolding properties and the fluid capacity of the manifold 220. Other materials may be used to form the manifold 220, such as, woven and non-woven materials, fibrous materials, non-woven Freudenberg M1545N or M1550, non-woven Texsus Multitex, and other similar materials.

The manifold 220 includes a first portion 222 configured to interface with the wound site 12 and a second portion 224 spaced apart from the wound site 12. Specifically, the second portion 224 of the manifold 220 extends from the first portion 222 of the manifold 220 along a longitudinal axis 221. Further, the manifold 220 extends along a transverse axis 223 perpendicular to the longitudinal axis 221.

In some embodiments, the first portion 222 of the manifold 220 includes a first maximum width 222W perpendicular to the longitudinal axis 221. In other words, the first portion 222 of the manifold 220 includes the first maximum width 222W along the transverse axis 223. In some embodiments, the second portion 224 of the manifold 220 includes a second maximum width 224W perpendicular to the longitudinal axis 221. In other words, the second portion 224 of the manifold 220 includes the second maximum width 224W along the transverse axis 223. In some embodiments, the first maximum width 222W is greater than the second maximum width 224W by a factor of at least two. In the illustrated embodiment of FIGS. 2A-2C, the first maximum width 222W is greater than the second maximum width 224W by a factor of four. However, in some other embodiments, the first maximum width 222W may be greater than the second maximum width 224W by any factor, as per desired application attributes.

In some embodiments, the first portion 222 of the manifold 220 includes a first length 222L along the longitudinal axis 221. In some embodiments, the second portion 224 of the manifold 220 includes a second length 224L along the longitudinal axis 221. In some embodiments, the second length 224L is greater than or equal to the first length 222L. In the illustrated embodiment of FIGS. 2A-2C, the second length 224L is greater than the first length 222L. However, in some other embodiments, the second length 224L may be equal to the first length 222L.

In the illustrated embodiment of FIGS. 2A-2C, the first portion 222 of the manifold 220 includes a circular shape and the second portion 224 of the manifold 220 includes a rectangular shape. In other embodiments, each of the first portion 222 and the second portion 224 may have a same shape, for example, a rectangular shape.

In some embodiments, the base film 210 includes a cutout 214 at least partially aligned with the manifold 220. In some embodiments, the cutout 214 is at least partially aligned with the first portion 222 of the manifold 220. In the illustrated embodiment of FIGS. 2A-2C, the cutout 214 is partially aligned with the first portion 222 and the second portion 224 of the manifold 220.

In some embodiments, a shape of the cutout 214 is similar to a shape of the manifold 220. In some embodiments, the shape of the cutout 214 is similar to the shape of the first portion 222 of the manifold 220. In the illustrated embodiment of FIGS. 2A-2C, the shape of the cutout 214 is similar to a combined shape of the first portion 222 and the second portion 224 of the manifold 220.

In some embodiments, the manifold 220 includes a manifold channel 226 extending at least partially along the manifold 220 and fluidly communicating with the wound site 12. In some embodiments, the manifold channel 226 extends along the longitudinal axis 221. In some embodiments, the manifold channel 226 extends at least partially along each of the first portion 222 of the manifold 220 and the second portion 224 of the manifold 220. In the illustrated embodiment of FIGS. 2A-2C, the manifold channel 226 extends entirely along the first portion 222 of the manifold 220 and partially along the second portion 224 of the manifold 220.

Further, the absorbent 230 is spaced apart from the first portion 222 of the manifold 220. In the illustrated embodiment of FIGS. 2A-2C, the absorbent 230 is spaced apart from the first portion 222 along the longitudinal axis 221. More particularly, the absorbent 230 is spaced apart from the first portion 222, such that a gap 231 exists between the absorbent 230 and the first portion 222. The absorbent 230 is coupled to and extends along the second portion 224 of the manifold 220, such that the absorbent 230 is offset from the wound site 12. More particularly, the absorbent 230 is offset from the wound site 12, such that a gap 233 exists between the absorbent 230 and the wound site 12. In some embodiments, the second length 224L of the second portion 224 may be at least twice of a length 230L of the absorbent 230. In the illustrated embodiment of FIGS. 2A-2C, the length 230L of the absorbent 230 may be between 80% to 85% of the second length 224L of the second portion 224. However, in alternative embodiments, the length 230L of the absorbent 230 may be less than 50% of the second length 224L of the second portion 224. Further, in some embodiments, the absorbent 230 is offset from the manifold channel 226. In the illustrated embodiment of FIGS. 2A-2C, the absorbent 230 is offset from the manifold channel 226 along the transverse axis 223 perpendicular to the longitudinal axis 221. Specifically, the absorbent 230 is offset from the manifold channel 226, such that a gap 235 exists between the manifold channel 226 and the absorbent 230.

The absorbent 230 may be configured to absorb liquids. Specifically, the absorbent 230 absorbs the wound exudate removed from the wound site 12. Since the absorbent 230 is spaced apart from the first portion 222 and is coupled to and extends along the second portion 224 of the manifold 220, a possibility of skin maceration, when the absorbent 230 is saturated with the wound exudate, may be substantially reduced. In other words, as the absorbent 230 is not localized, or disposed at the wound site 12 shown in FIG. 1, a possibility of skin maceration, when the absorbent 230 is saturated with the wound exudate, may be reduced.

In an embodiment, a material of the absorbent 230 may include BASF Luquafleece 402C. However, other materials may be used to form the absorbent 230, for example, superabsorbent polymers disposed on woven and non-woven substrates, fibrous materials, non-woven superabsorbent fiber by Technical Absorbents Limited, non-woven Texsus Absortex, and the like.

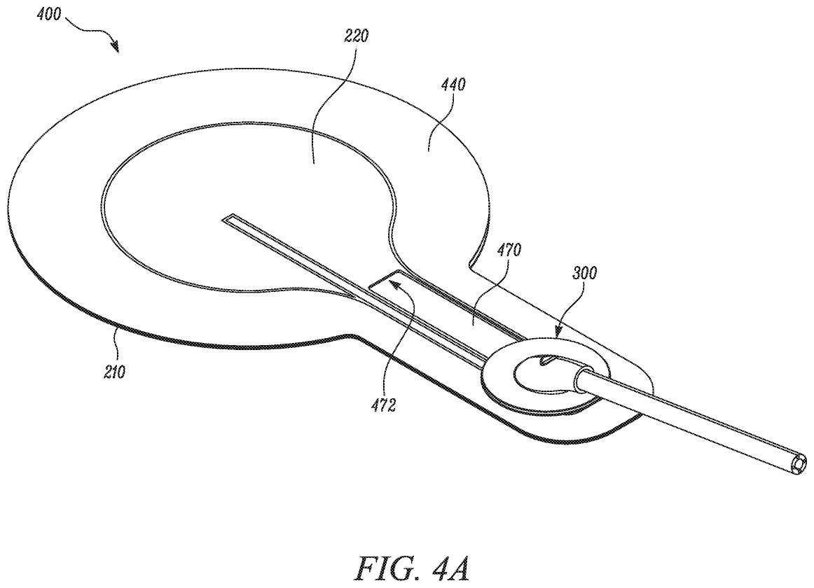

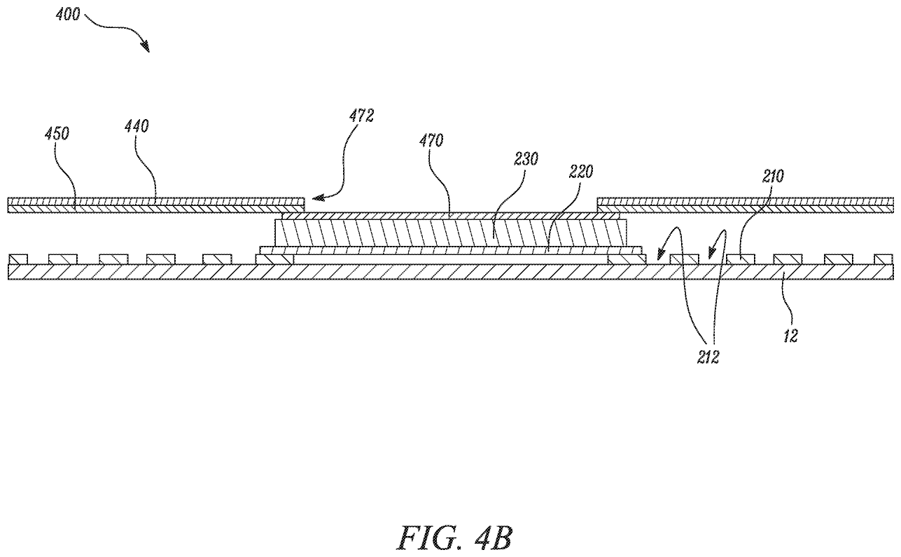

Further, the top film 240 is disposed adjacent to the absorbent 230 opposite to the manifold 220, such that the absorbent 230 is disposed between the manifold 220 and the top film 240. In some embodiments, the top film 240 is fluid permeable. In some embodiments, the top film 240 may include an adhesive coating (such as, an adhesive coating 450 as shown in FIG. 4B). The adhesive coating may be at least partially disposed between the top film 240 and the base film 210. The adhesive coating may pass through the perforations 212 and allow removable connection of the dressing device 200 with the skin of the user. In some embodiments, the adhesive coating may include a patient friendly adhesive as it interacts with the skin of the user. In an embodiment, the adhesive coating may include an acrylic adhesive. In some embodiments, the adhesive coating may include pattern coated adhesives that may enhance evaporation of the water content from the wound exudate and improve total fluid handling.

In some embodiments, the top film 240 and the base film 210 may be at least partially connected to each other. In some embodiments, the top film 240 and the base film 210 may be at least partially connected to each other via the adhesive coating. In other embodiments, the top film 240 and the base film 210 may be at least partially connected to each other by a heat seal, a weld seam, bonding, stitching, and the like.

In an example, the top film 240 may be made of an elastomeric material. Examples of the elastomeric material may include, but is not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane, ethylene vinyl acetate (EVA) film, co-polyester, and silicones.

In some embodiments, the top film 240 includes a top channel 242 extending along the top film 240 and aligned with the manifold channel 226. In some embodiments, the dressing device 200 further includes a top cover 244 disposed between the manifold 220 and the top film 240. The top cover 244 is coupled to the manifold 220 and extends along the manifold channel 226, such that the top cover 244 and the manifold channel 226 together define a pressure sensing pathway 250 therebetween. The pressure sensing pathway 250 is depicted by arrows herein. In the illustrated embodiments of FIG. 2A-2C, pressure sensing occurs under the absorbent 230, such that the NPWT system 100 (see FIG. 1) may be able to ascertain whether the dressing device 200 is full. Further, the pressure sensing under the absorbent 230 may ensure accurate pressure monitoring. The pressure sensing pathway 250 may allow the pressure sensing device 104 (see FIG. 1) to fluidly communicate with a region under the absorbent 230 in order to determine a level of saturation of the absorbent 230. In other words, the pressure sensing device 104 together with the dressing device 200 may be used to determine if the dressing device 200 is full so that the dressing device 200 may be removed/replaced.

In the illustrated embodiment of FIGS. 2A-2C, the top channel 242 is configured to at least partially receive the top cover 244 therewithin. Therefore, in some embodiments, a shape of the top cover 244 is similar to a shape of the top channel 242. The top cover 244 may be welded to the manifold 220 or the top film 240. In an example, the top cover 244 may be made of an elastomeric material. Examples of the elastomeric material may include, but is not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane, ethylene vinyl acetate (EVA) film, co-polyester, and silicones.

In some embodiments, the dressing device 200 further includes a gasket 260 at least partially received within each of the absorbent 230 and the top film 240. The gasket 260 may be made from, for example, rubber or silicone, without any limitations. In some embodiments, the gasket 260 includes a gasket opening 262 therethrough disposed in fluid communication with the manifold channel 226. The gasket opening 262 is in fluid communication with the pressure sensing pathway 250.

In some embodiments, the absorbent 230 includes an absorbent cutout 232. In some embodiments, the top film 240 includes a through-hole 246 at least partially aligned with the absorbent cutout 232. The gasket 260 is at least partially received within each of the absorbent cutout 232 and the through-hole 246.

In some embodiments, the dressing device 200 further includes a pad assembly 300 coupled to the top film 240. In an example, the pad assembly 300 may include a Sensa T.R.A.C.™ pad available from 3M. The pad assembly 300 includes a tubing 306 and a connector 308 that together connect the dressing device 200 with the negative pressure source 102 and the pressure sensing device 104.

In some embodiments, the pad assembly 300 includes a pressure sensing channel 302 disposed in fluid communication with the pressure sensing pathway 250 and configured to fluidly communicate with the pressure sensing device 104 spaced apart from the top film 240. Specifically, in the illustrated embodiment of FIGS. 2A-2C, the pad assembly 300 includes the pressure sensing channel 302 disposed in fluid communication with the gasket opening 262 and configured to fluidly communicate with the pressure sensing device 104 spaced apart from the top film 240.

In some embodiments, the pad assembly 300 further includes a negative pressure channel 304 fluidly isolated from the pressure sensing channel 302 and disposed in fluid communication with the top film 240 and configured to fluidly communicate with the negative pressure source 102. In some embodiments, the manifold 220, the absorbent 230, and the top film 240 together form a negative pressure pathway 252 fluidly communicating the wound site 12 with the negative pressure channel 304. The negative pressure pathway 252 is depicted by arrows herein.

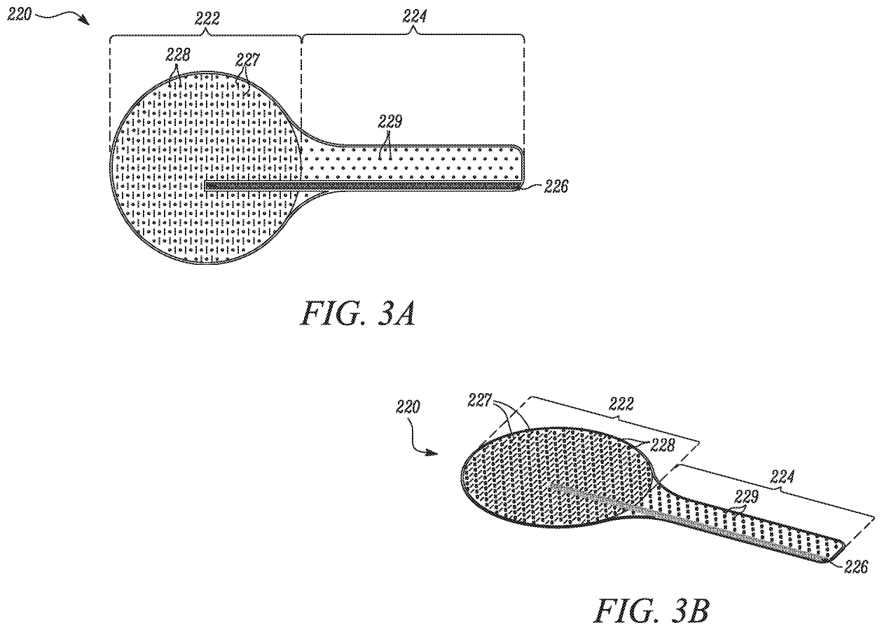

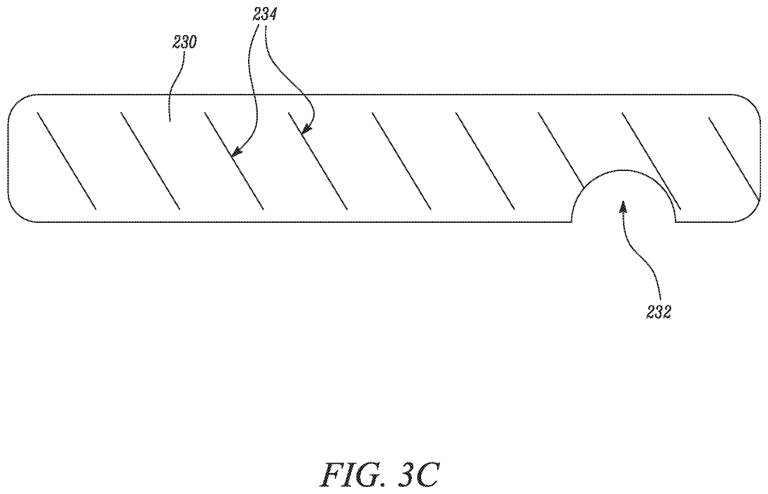

FIGS. 3A and 3B illustrate the manifold 220 of the dressing device 200 shown in FIGS. 2A-2C, according to an embodiment of the present disclosure. Specifically, FIG. 3A illustrates a schematic top view of the manifold 220. FIG. 3B illustrates a schematic side perspective view of the manifold 220.

As shown in FIGS. 3A and 3B, in some embodiments, the first portion 222 of the manifold 220 includes a plurality of perforations 227. The plurality of perforations 227 may take many shapes or combinations of shapes, including circular apertures, elliptical apertures, rectangular openings, or polygons, for example. In the illustrated embodiment of FIGS. 3A and 3B, each of the plurality of perforations 227 is a slit, or a linear cut. The plurality of perforations 227 may provide fluid communication of the first portion 222 of the manifold 220 with the wound site 12 (see FIG. 1).

In some embodiments, the first portion 222 of the manifold 220 further includes a plurality of projections 228 extending opposite to the base film 210 (see FIG. 2B). In the illustrated embodiment of FIGS. 3A and 3B, each of the plurality of projections 228 has a circular cross-section. Alternatively, each of the plurality of projections 228 may have a square cross-section, a rectangular cross-section, an oval cross-section, a triangular cross-section, and the like.

In some embodiments, the plurality of perforations 227 in the first portion 222 alternates with the plurality of projections 228 in the first portion 222. However, the plurality of perforations 227 in the first portion 222 and the plurality of projections 228 in the first portion 222 may be arranged in any suitable manner, as per desired application attributes.

In some embodiments, the second portion 224 of the manifold 220 includes a plurality of projections 229 extending opposite to the base film 210 and at least partially engaging the absorbent 230 shown in FIGS. 2A-2C. In the illustrated embodiment of FIGS. 3A and 3B, each of the plurality of projections 229 has a circular cross-section. Alternatively, each of the plurality of projections 229 may have a square cross-section, a rectangular cross-section, an oval cross-section, a triangular cross-section, and the like. The plurality of projections 228, 229 may improve distribution of the negative pressure along the negative pressure pathway 252 (see FIG. 2C). It should be noted that the projections 228, 229 are absent from the manifold channel 226.

Further, in some examples, when the absorbent 230 is saturated, the plurality of projections 229 of the the second portion 224 of the manifold 220 may engage with the absorbent 230. This way, the plurality of projections 229 of the the second portion 224 of the manifold 220 may define the negative pressure pathway 252 even when the absorbent 230 is saturated with the wound exudate. Thus, the dressing device 200 may allow effective management of the wound exudate in all orientations and may reduce a possibility of blocking of the negative pressure pathway 252 when the absorbent 230 is saturated with the wound exudate.

Further, the manifold 220 does not include any perforations in the the second portion 224. This may prevent leakage of the wound exudate from the absorbent 230 to the skin of the user.

FIG. 3C illustrates a schematic top view of the absorbent 230 of the dressing device 200 shown in FIGS. 2A-2C, according to an embodiment of the present disclosure.

As shown in FIG. 3C, in some embodiments, the absorbent 230 further includes a plurality of perforations 234. However, in some other embodiments, the absorbent 230 may omit the plurality of perforations 234.

The plurality of perforations 234 may take many shapes or combinations of shapes, including circular apertures, elliptical apertures, rectangular openings, or polygons, for example. In the illustrated embodiment of FIG. 3C, each of the plurality of perforations 234 is a slit, or a linear cut.

FIGS. 4A and 4B illustrate a dressing device 400 for the NPWT system 100 of FIG. 1, according to another embodiment of the present disclosure. FIG. 4A illustrates a schematic perspective view of the dressing device 400. FIG. 4B illustrates a schematic partial sectional side view of the dressing device 400. The dressing device 400 is functionally equivalent to the dressing device 200 of FIGS. 2A to 3C. Same parts are referred to herein by same numbers.

As shown in FIG. 4A, the dressing device 400 includes a top film 440. The top film 440 is substantially similar to the top film 240. However, a portion of the top film 440 further includes a high moisture vapor transmission rate (MVTR) film 470. As shown in FIG. 4B, the top film 440 may include a slot 472 such that the MVTR film 470 is in alignment with the slot 472. In some embodiments, the high MVTR film 470 of the top film 440 is at least partially aligned with the absorbent 230. In some examples, the high MVTR film 470 may include foams, such as, polyurethane foam, Libeltex TDL2, Libeltex TL4, Baltex 3DXD spacer fabrics, Baltex 4DXD spacer fabrics, or the like.

In some embodiments, the dressing device 400 may include an adhesive coating 450 at least partially disposed between the top film 440 and the base film 210. In some embodiments, the adhesive coating 450 is absent from the region that includes the MVTR film 470. In other words, the portion of the top film 440 including the high MVTR film 470 may be substantially free of the adhesive coating 450. The adhesive coating 450 may be configured to removably connect the dressing device 400 with the skin of the user. In some embodiments, the adhesive coating 450 may include an acrylic adhesive.

Typically, the adhesive coating 450 negatively affects the evaporation of the water content from the wound exudate. The high MVTR film 470 may increase total fluid handling volume by utilizing evaporation to a greater extent. In other words, the high MVTR film 470 may improve management of the wound exudate by evaporating off more of the water content from the wound exudate. In turn, the dressing device 400 may be used for longer durations.

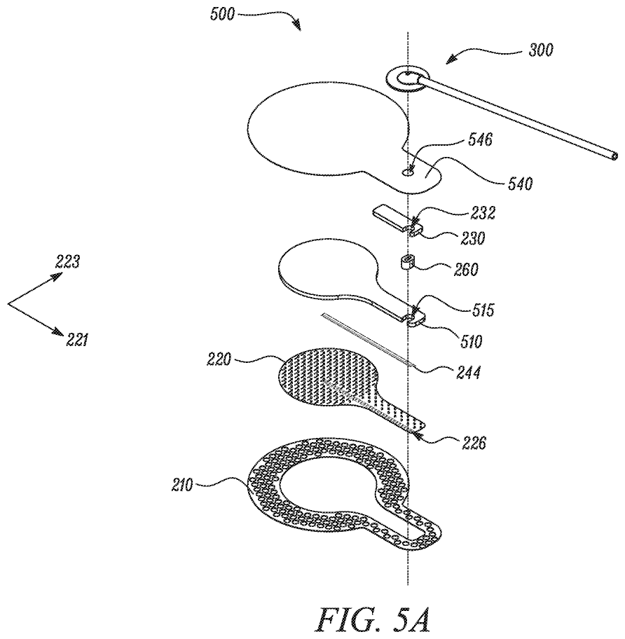

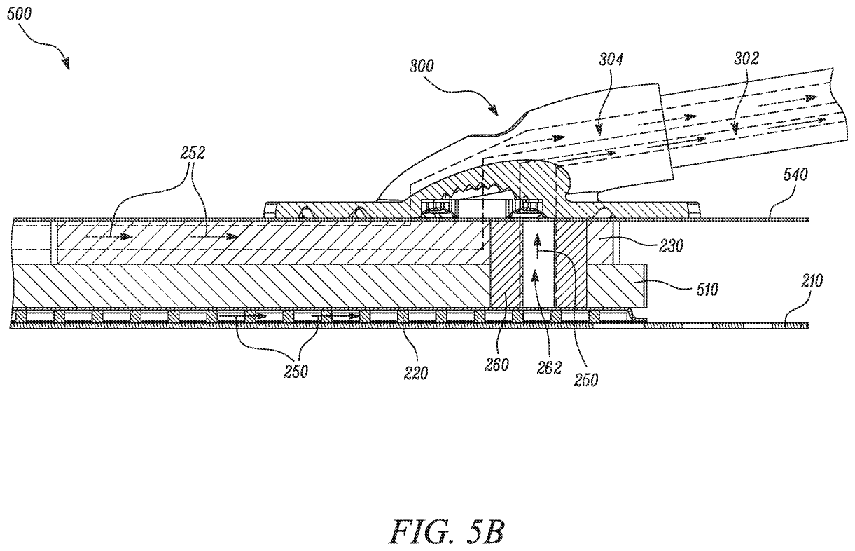

FIGS. 5A and 5B illustrate a dressing device 500 for the NPWT system 100 of FIG. 1, according to another embodiment of the present disclosure. FIG. 5A illustrates a schematic exploded perspective view of the dressing device 500. FIG. 5B illustrates a schematic partial sectional side view of the dressing device 500. The dressing device 500 is functionally equivalent to the dressing device 200 shown in FIGS. 2A-2C. Same parts are referred to herein by same numbers.

The dressing device 500 includes a top film 540. The top film 540 is substantially similar to the top film 240 shown in FIGS. 2A-2C. However, the top film 540 does not include the top channel 242 (see FIG. 2B).

As shown in FIGS. 5A and 5B, the dressing device 500 further includes an intermediate manifold 510 disposed between the manifold 220 and the absorbent 230. In some embodiments, the intermediate manifold 510 and the manifold 220 have the same shape and dimensions. Further, the top cover 244 is disposed between the manifold 220 and the intermediate manifold 510. The intermediate manifold 510 may ensure even distribution of the negative pressure along the wound site 12 (see FIG. 1). In some embodiments, the intermediate manifold 510 includes an open cell, reticulated polyurethane foam, such as, GRANUFOAM™ Dressing or VERAFLO™ Therapy foam, both available from KCI Licensing, Inc. of San Antonio, Tex. In other embodiments, the intermediate manifold 510 may include foams, such as, polyurethane foam, Libeltex TDL2, Libeltex TL4, Baltex 3DXD spacer fabrics, Baltex 4DXD spacer fabrics, or the like.

As shown in FIGS. 5A and 5B, the gasket 260 is at least partially received through the intermediate manifold 510. Specifically, the intermediate manifold 510 includes an intermediate manifold cutout 515 at least partially receiving the gasket 260 therethrough.

In some embodiments, the top film 540 further includes a through-hole 546 at least partially aligned with the absorbent cutout 232. The gasket 260 is at least partially received within each of the intermediate manifold cutout 515, the absorbent cutout 232, and the through-hole 546.

It should be noted that details related to the negative pressure pathway 252, the pressure sensing pathway 250, the negative pressure channel 304, and the pressure sensing channel 302 as described in relation to FIGS. 2A-2C are equally applicable to the dressing device 500 of FIGS. 5A and 5B.

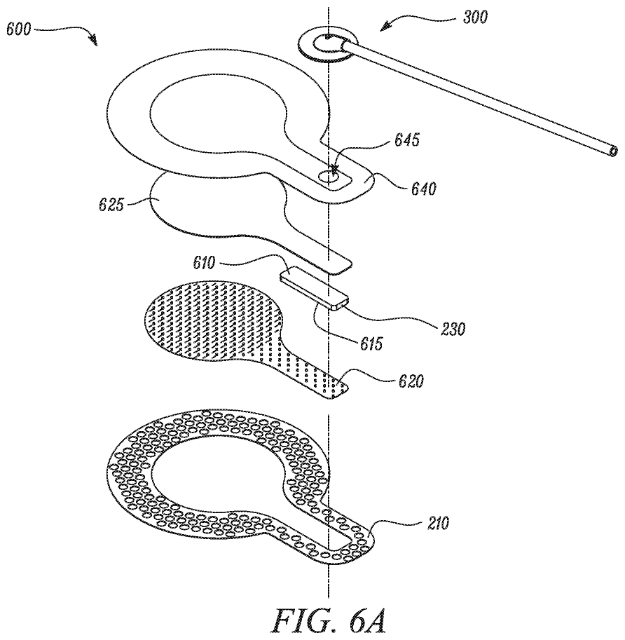

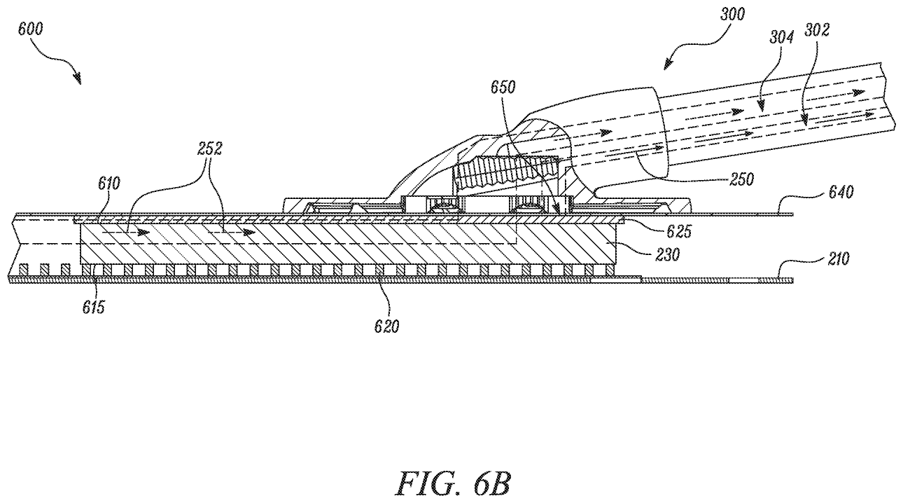

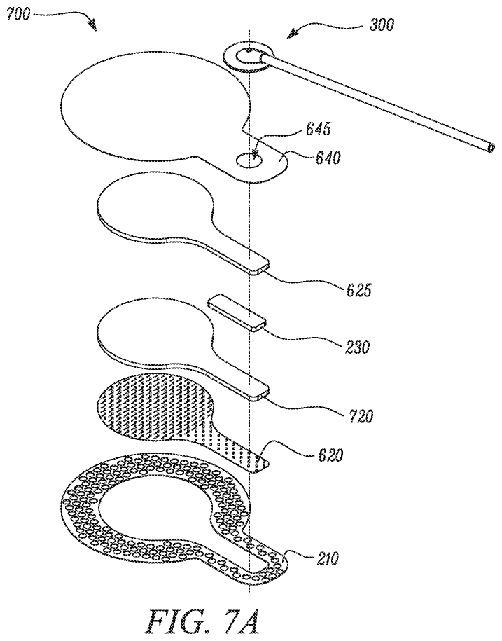

FIGS. 6A and 6B illustrate a dressing device 600 for the NPWT system 100 of FIG. 1, according to another embodiment of the present disclosure. FIG. 6A illustrates a schematic exploded perspective view of the dressing device 600. FIG. 6B illustrates a schematic partial sectional side view of the dressing device 600. The dressing device 600 is functionally equivalent to the dressing device 200 shown in FIGS. 2A-2C. Same parts are referred to herein by same numbers.

The dressing device 600 includes a top film 640. The top film 640 is substantially similar to the top film 240 shown in FIGS. 2A-2C. However, the top film 640 does not include the top channel 242 (see FIG. 2B).

The dressing device 600 includes a manifold 620 substantially similar to the manifold 220 shown in FIGS. 2A-2C. However, the manifold 620 does not include the manifold channel 226 (see FIGS. 3A and 3B).

Further, in the illustrated embodiment of FIGS. 6A and 6B, the dressing device 600 does not include the gasket 260 (see FIGS. 2B and 2C). Therefore, the absorbent 230 does not include the absorbent cutout 232 (see FIG. 2B).

The absorbent 230 includes a first major surface 610 disposed adjacent to the top film 640 and a second major 615 surface opposite to the first major surface 610. The first major surface 610 of the absorbent 230 forms a pressure sensing region 650. In this embodiment, the pressure sensing occurs above the absorbent 230, which may simplify a design of the dressing device 600.

In some embodiments, the top film 640 includes a through-hole 645 aligned with and fluidly communicating with the pressure sensing region 650 of the absorbent 230.

In the illustrated embodiment of FIGS. 6A and 6B, the pad assembly 300 includes the pressure sensing channel 302 disposed in fluid communication with the through-hole 645 of the top film 640 and configured to fluidly communicate with the pressure sensing device 104 (see FIG. 1) spaced apart from the top film 640. The pad assembly 300 further includes the negative pressure channel 304 fluidly isolated from the pressure sensing channel 302 and disposed in fluid communication with the top film 640 and configured to fluidly communicate with the negative pressure source 102 (see FIG. 1). The manifold 620, the absorbent 230, and the top film 640 together form the negative pressure pathway 252 fluidly communicating the wound site 12 (see FIG. 1) with the negative pressure channel 304.

As shown in FIG. 6B, in some embodiments, the dressing device 600 includes an additional manifold 625 disposed between the top film 640 and the absorbent 230. The additional manifold 625 may ensure even distribution of the negative pressure along the wound site 12. In some embodiments, the additional manifold 625 may be formed from Libeltex TDL2 having a material weight of 80 grams per square member (gsm). In other embodiments, the additional manifold 625 may have a material weight between about 20 gsm and about 140 gsm. Larger material weights may be selected to increase manifolding properties and the fluid capacity of the additional manifold 625. Other materials may be used to form the additional manifold 625, such as, woven and non-woven materials, fibrous materials, non-woven Freudenberg M1545N or M1550, non-woven Texsus Multitex, and other similar materials.

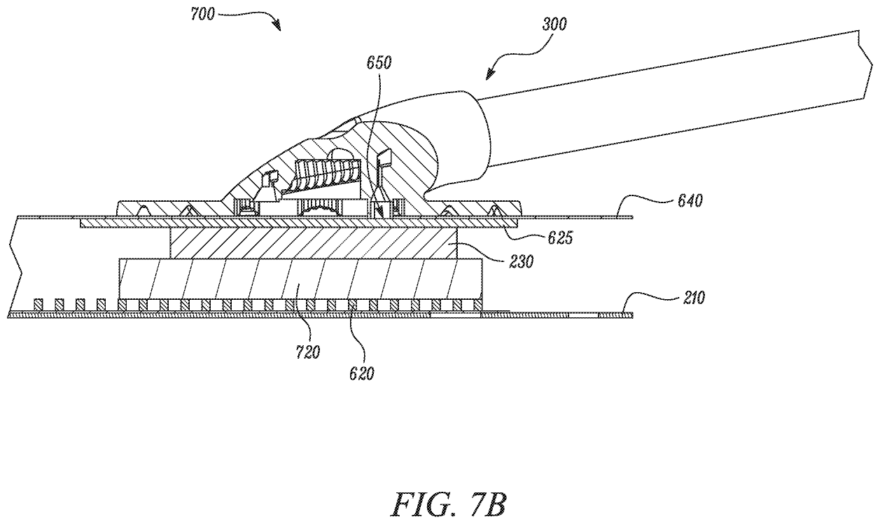

FIGS. 7A and 7B illustrate a dressing device 700 for the NPWT system 100 of FIG. 1, according to another embodiment of the present disclosure. FIG. 7A illustrates a schematic exploded perspective view of the dressing device 700. FIG. 7B illustrates a schematic partial sectional side view of the dressing device 700. The dressing device 700 is functionally equivalent to the dressing device 600 shown in FIGS. 6A and 6B. Same parts are referred to herein by same numbers.

In some embodiments, the dressing device 700 includes an intermediate manifold 720 disposed between the manifold 620 and the absorbent 230. In some embodiments, the intermediate manifold 720 and the manifold 620 have the same shape and dimensions. Further, a material of the intermediate manifold 720 may be same as the material of the intermediate manifold 510. The dressing device 700 further includes the additional manifold 625 disposed between the top film 640 and the absorbent 230. The additional manifold 625 and the intermediate manifold 720 may ensure even distribution of the negative pressure along the wound site 12 (see FIG. 1).

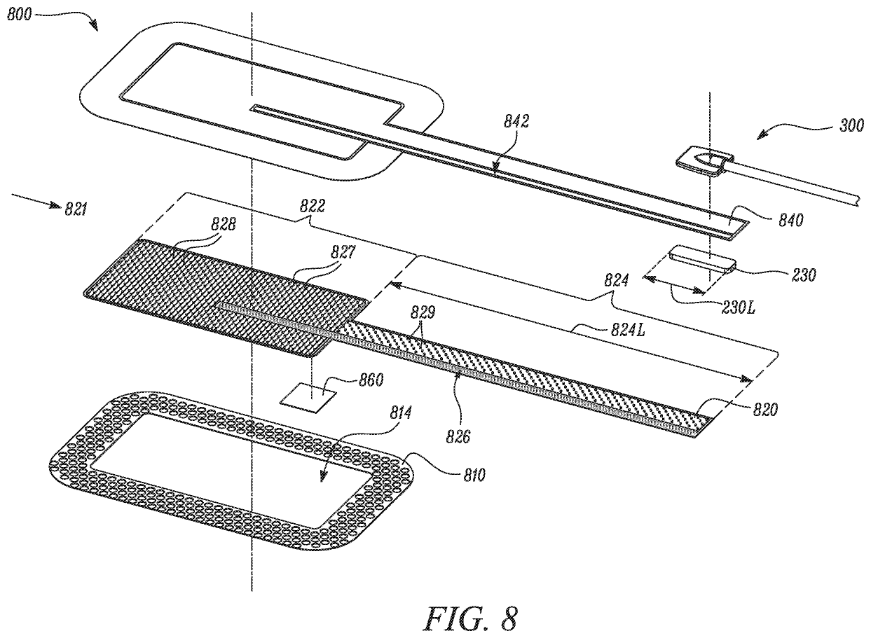

FIG. 8 illustrates a dressing device 800 for the NPWT system 100 of FIG. 1, according to another embodiment of the present disclosure. Specifically, FIG. 8 illustrates a schematic exploded perspective view of the dressing device 800. The dressing device 800 is functionally equivalent to the dressing device 200 shown in FIGS. 2A-2C. Same parts are referred to herein by same numbers.

The dressing device 800 includes a base film 810, a manifold 820, and a top film 840 functionally equivalent to the base film 210, the manifold 220, and the top film 240 (see FIGS. 2A-2C), respectively of the dressing device 200. Further, materials of the base film 810, the manifold 820, and the top film 840 are same as the materials of the base film 210, the manifold 220, and the top film 840, respectively of the dressing device 200. The base film 810 is substantially rectangular in shape. The base film 810 defines a cutout 814. Further, in the illustrated embodiment of FIG. 8, the manifold 820 includes a first portion 822 and a second portion 824. Each of the first portion 822 and the second portion 824 includes a rectangular shape herein. The first portion 822 includes a plurality of perforations 827 and a plurality of projections 828 alternating with the plurality of perforations 827. Further, the second portion 824 includes a plurality of projections 829. The projections 828, 829 may define a negative pressure pathway (not shown in the accompanying figure) for applying the negative pressure even when the absorbent 230 is saturated with the wound exudate. It should be noted that the projections 828, 829 are also present in the manifold channel 826.

Further, the cutout 814 is partially aligned with the manifold 820. Specifically, the cutout 814 is only in alignment with the first portion 822 of the manifold 820. The shape of the cutout 814 is similar to the shape of the first portion 822 of the manifold 820.

Further, the manifold 820 includes a manifold channel 826 extending at least partially along the manifold 820 and fluidly communicating with the wound site 12 (see FIG. 1). In some embodiments, the manifold channel 826 extends along a longitudinal axis 821. In some embodiments, the manifold channel 826 extends at least partially along each of the first portion 822 of the manifold 820 and the second portion 824 of the manifold 820. In the illustrated embodiment of FIG. 8, the manifold channel 826 extends entirely along the first portion 822 of the manifold 820 and partially along the second portion 824 of the manifold 820.

In some embodiments, the top film 840 includes a top channel 842 extending along the top film 840 and aligned with the manifold channel 826. In some embodiments, the manifold channel 826 and the top channel 842 together define a pressure sensing pathway (not shown) therebetween. Therefore, the pressure sensing occurs under the absorbent 230 and proximal to the wound site 12, such that the NPWT system 100 (see FIG. 1) may be able to ascertain whether the dressing device 800 is full. Further, the pressure sensing under the absorbent 230 may ensure accurate pressure monitoring. The pressure sensing pathway may allow the pressure sensing device 104 (see FIG. 1) to fluidly communicate with a region under the absorbent 230 in order to determine a level of saturation of the absorbent 230. In other words, the pressure sensing device 104 together with the dressing device 800 may be used to determine if the dressing device 800 is full so that the dressing device 800 may be removed/replaced.

In the illustrated embodiment of FIG. 8, the dressing device 800 further includes an adhesive layer 860 disposed between the manifold 820 and the base film 810. In some embodiments, the adhesive layer 860 may remove a leak path between the manifold 820 and the base film 810. In some examples, the adhesive layer 860 may include a medically acceptable adhesive, such as a pressure-sensitive adhesive, that extends about a portion of, a periphery of, or about all of the adhesive layer 860. In other examples, adhesive layer 860 may be a double-sided drape tape, a paste, a hydrocolloid, a hydrogel, a silicone gel, an organogel, or other sealing devices or elements. The adhesive layer 850 includes a square shape herein. Alternatively, the adhesive layer 860 may include any other shape.

Furthermore, in the illustrated embodiment of FIG. 8, the dressing device 800 does not include the gasket 260 (see FIGS. 2B and 2C). Therefore, the absorbent 230 does not include the absorbent cutout 232 (see FIG. 2A). Further, in the illustrated embodiment of FIG. 8, the length 230L of the absorbent 230 may be less than 50% of a second length 824L of the second portion 824. In an example, the length 230L of the absorbent 230 may be between 10% and 25% of the second length 224L of the second portion 824.

Referring to FIGS. 1 to 8, the offset between the absorbent 230 and the wound site 12 may prevent a direct contact between any wound exudate absorbed by the absorbent 230 and the wound site 12. Further, since the absorbent 230 is offset from the wound site 12, a possibility of skin maceration, when the absorbent 230 is saturated with the wound exudate, may be reduced.

The dressing device 200, 400, 500, 600, 700, 800 described herein may allow effective management of the wound exudate in all orientations and may reduce a possibility of blocking of negative pressure pathways when the absorbent 230 is saturated with the wound exudate.

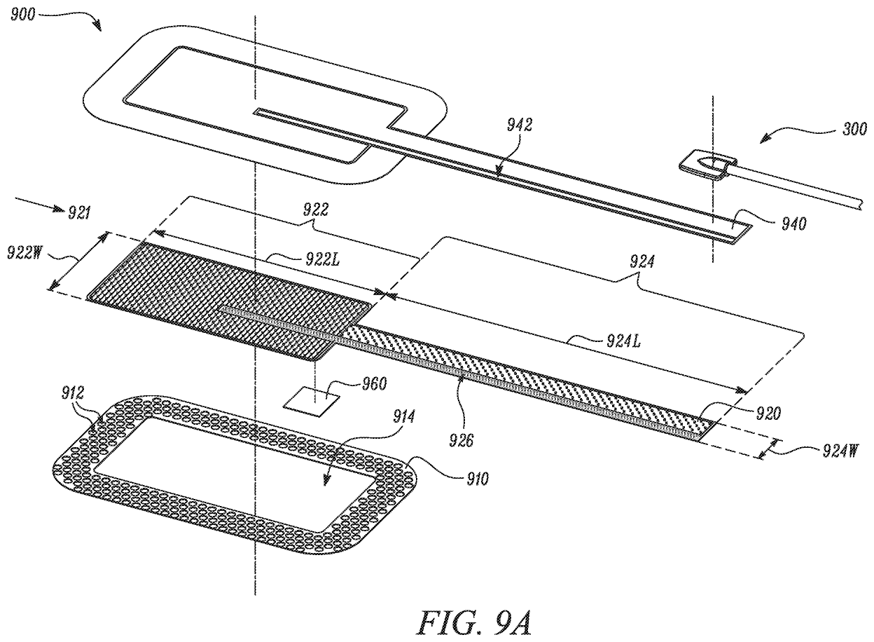

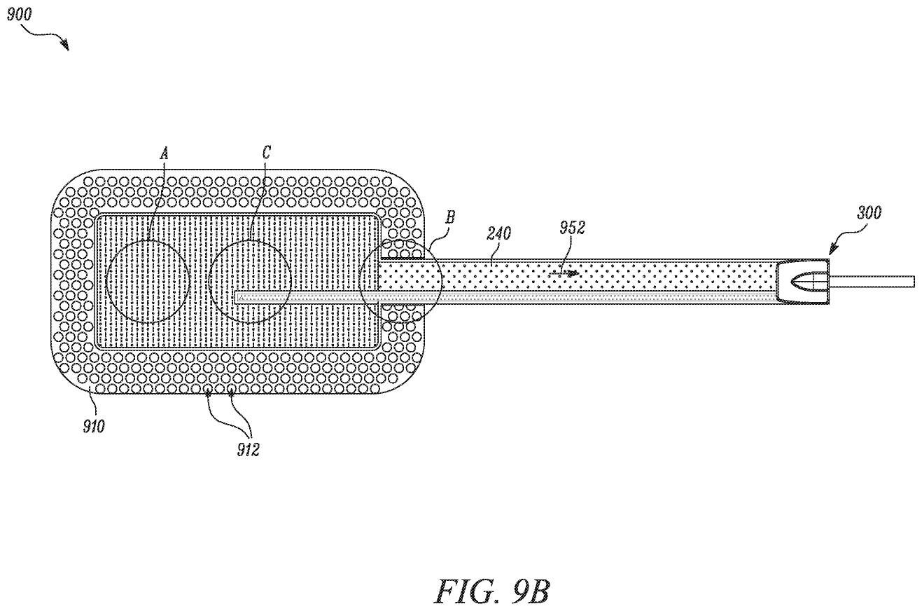

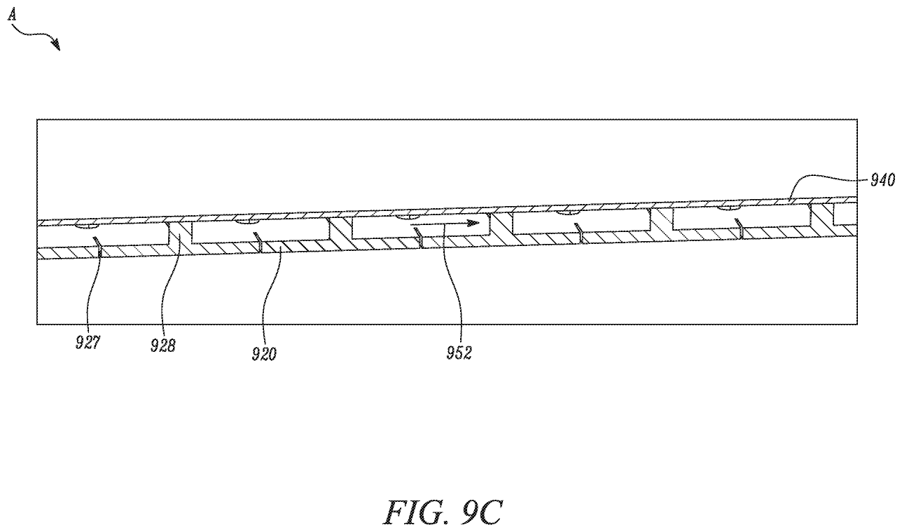

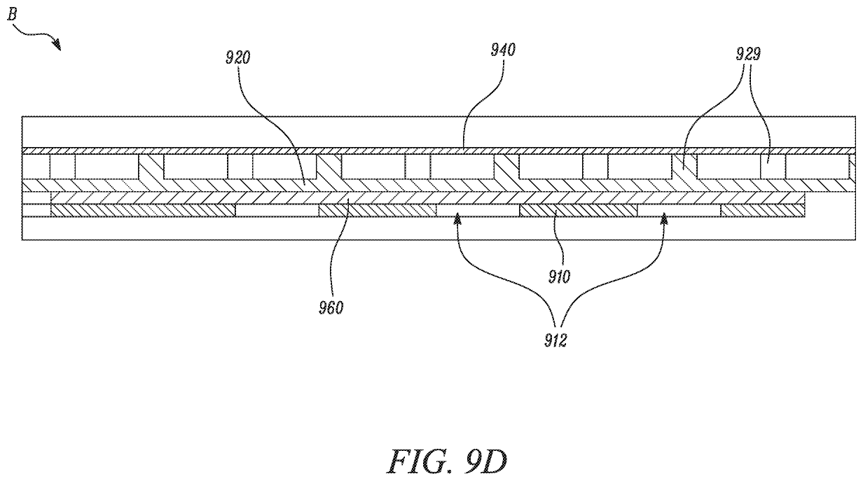

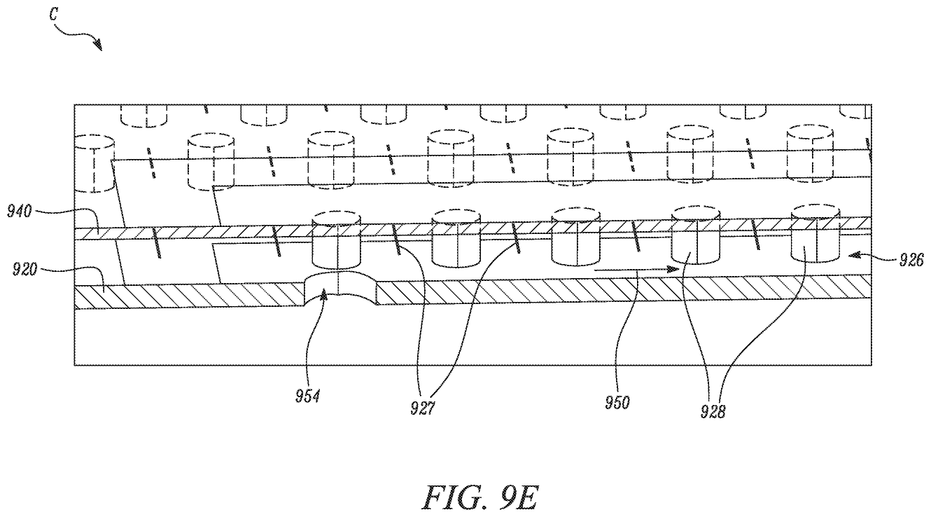

FIG. 9A illustrates a schematic exploded perspective view of a dressing device 900 for the NPWT system 100 of FIG. 1, according to another embodiment of the present disclosure. FIG. 9B illustrates a schematic top view of the dressing device 900, according to an embodiment of the present disclosure. FIG. 9C illustrates a magnified schematic sectional perspective view of a portion A of the dressing device 900 of FIG. 9B, according to an embodiment of the present disclosure. FIG. 9D illustrates a magnified schematic sectional perspective view of a portion B of the dressing device 900 of FIG. 9B, according to an embodiment of the present disclosure. FIG. 9E illustrates a magnified schematic sectional side view of a portion C of the dressing device 900 of FIG. 9B, according to an embodiment of the present disclosure.

The dressing device 900 is functionally equivalent to the dressing device 200 shown in FIGS. 2A-2C. However, the dressing device 900 does not include any absorbent (such as, the absorbent 230 shown in FIGS. 2A-2C). As shown in FIGS. 9A and 9B, the dressing device 900 includes a base film 910 configured to couple to the wound site 12 (see FIG. 1), and a manifold 920 coupled to the base film 910. The base film 910 is substantially similar and functionally equivalent to the base film 210 of FIGS. 2A-2C. The base film 910 includes a plurality of perforations 912. Further, the base film 910 includes a cutout 914 at least partially aligned with the manifold 920.

The manifold 920 includes a first portion 922 configured to interface with the wound site 12 and a second portion 924 spaced apart from the wound site 12. In the illustrated embodiment of FIG. 9A, the cutout 914 is at least partially aligned with the first portion 922 of the manifold 920. Further, a shape of the cutout 914 is similar to a shape of the first portion 922 of the manifold 920. The manifold 920 includes a transparent material. The manifold 920 further includes a manifold channel 926 extending along the manifold 920 and fluidly communicating with the wound site 12. The manifold channel 926 extends at least partially along each of the first portion 922 of the manifold 920 and the second portion 924 of the manifold 920. Specifically, the manifold channel 926 extends entirely along each of the first portion 922 of the manifold 920 and partially along the second portion 924 of the manifold 920.

In some embodiments, the manifold channel 926 extends along a longitudinal axis 921. The first portion 922 of the manifold 920 may include a first maximum width 922W perpendicular to the longitudinal axis 921 and the second portion 924 of the manifold 920 may include a second maximum width 924W perpendicular to the longitudinal axis 921. In some embodiments, the first maximum width 922W is greater than the second maximum width 924W by a factor of at least two.

In some embodiments, the first portion 922 of the manifold 920 includes a first length 922L along the longitudinal axis 921 and the second portion 924 of the manifold 920 includes a second length 924L along the longitudinal axis 921. In some embodiments, the second length 924L is greater than or equal to the first length 922L. In the illustrated embodiment of FIG. 9A, the second length 924L is greater than the first length 922L.

The dressing device 900 further includes a top film 940 disposed adjacent to the manifold 920 opposite to the base film 910, such that the manifold 920 is disposed between the base film 910 and the top film 940. In other words, the top film 940 is disposed directly adjacent to the manifold 920 with no intervening components or films (e.g., the absorbent 230). The top film 940 includes a top channel 942 extending along the top film 940 and aligned with the manifold channel 926. Further, materials of the base film 910, the manifold 920, and the top film 940 are same as the materials of the base film 210, the manifold 220, and the top film 240 of the dressing device 200, respectively.

The dressing device 900 further includes an adhesive layer 960 disposed between the manifold 920 and the base film 910. In some embodiments, the adhesive layer 960 may remove a leak path between the manifold 920 and the base film 910. In some examples, the adhesive layer 960 may include a medically acceptable adhesive, such as a pressure-sensitive adhesive, that extends about a portion of, a periphery of, or about all of the adhesive layer 960. In other examples, adhesive layer 960 may be a double-sided drape tape, a paste, a hydrocolloid, a hydrogel, a silicone gel, an organogel, or other sealing devices or elements.

As shown in FIG. 9C, the first portion 922 of the manifold 920 includes a plurality of perforations 927. Referring to FIGS. 9C and 9D, each of the first portion 922 and the second portion 924 of the manifold 920 includes a plurality of projections 928, 929, respectively, extending opposite to the base film 910 and at least partially engaging the top film 940. The plurality of perforations 927 in the first portion 922 alternates with the plurality of projections 928 in the first portion 922. As shown in FIGS. 9C and 9E, the projections 928, 929 are also present in the manifold channel 926.

Referring now to FIGS. 9A to 9E, the manifold 920 and the top film 940 together form a negative pressure pathway 952 fluidly communicating the wound site 12 (see FIG. 1) with the negative pressure channel 304 (see FIG. 9C). Further, in the illustrated embodiment of FIGS. 9A to 9E, the manifold channel 926 and the top channel 942 together define a pressure sensing pathway 950 therebetween. In some embodiments, the first portion 922 of the manifold 920 defines a through-opening 954 such that the through-opening 954 fluidly communicates the pressure sensing pathway 950 with the wound site 12. Therefore, in this embodiment, the pressure sensing occurs directly above the wound site 12.

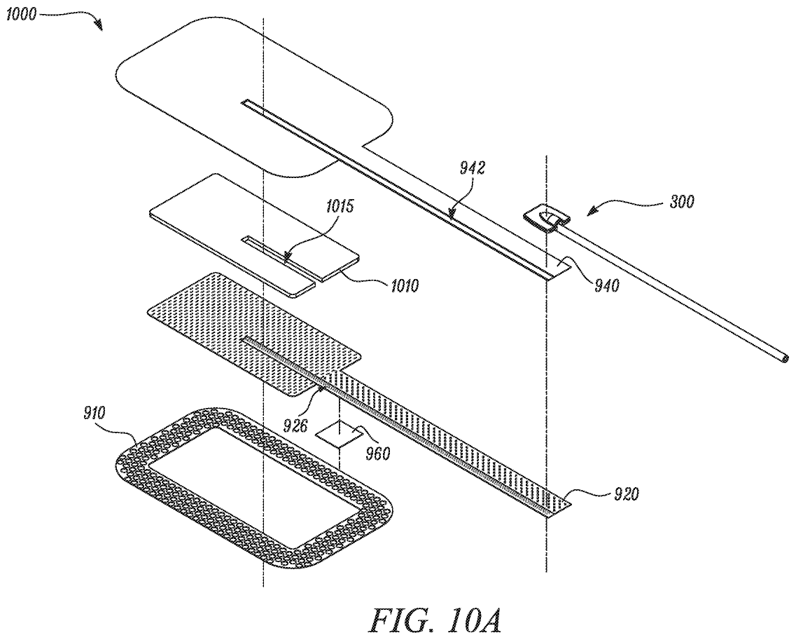

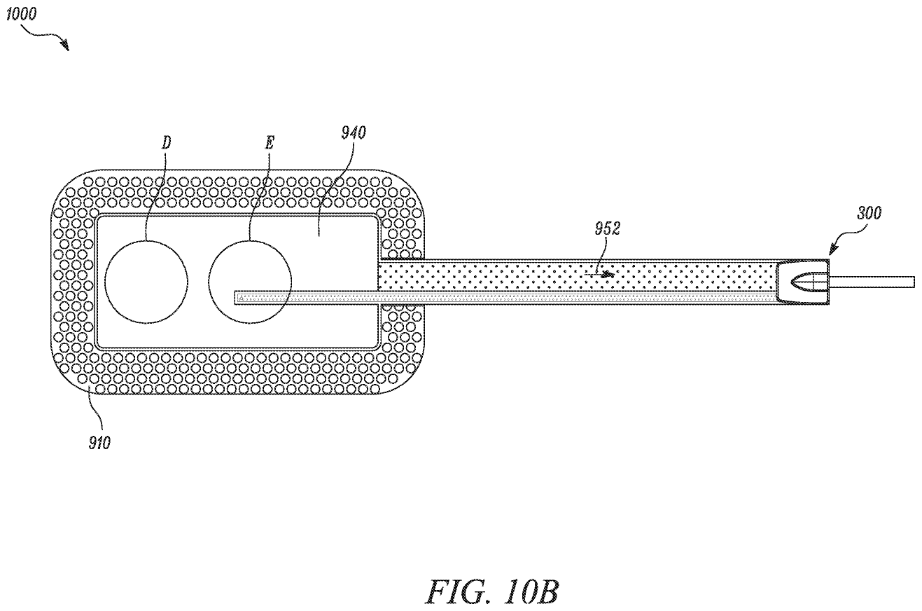

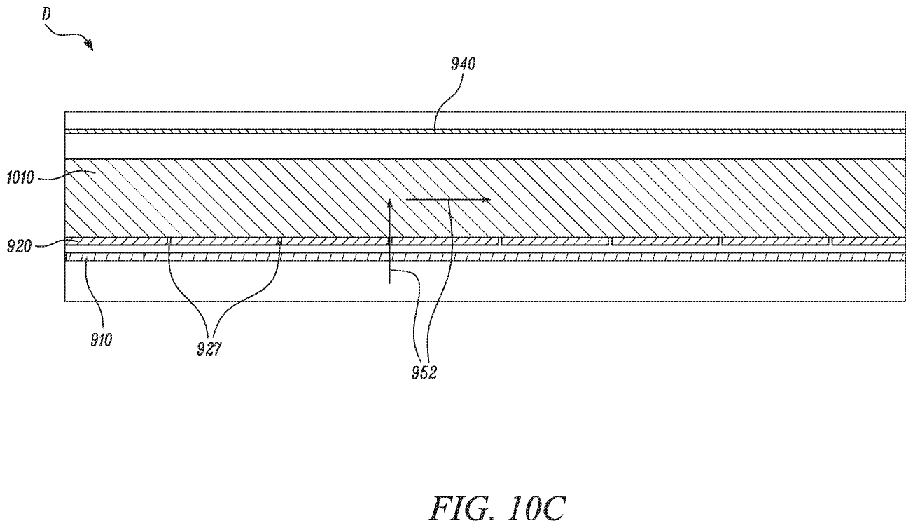

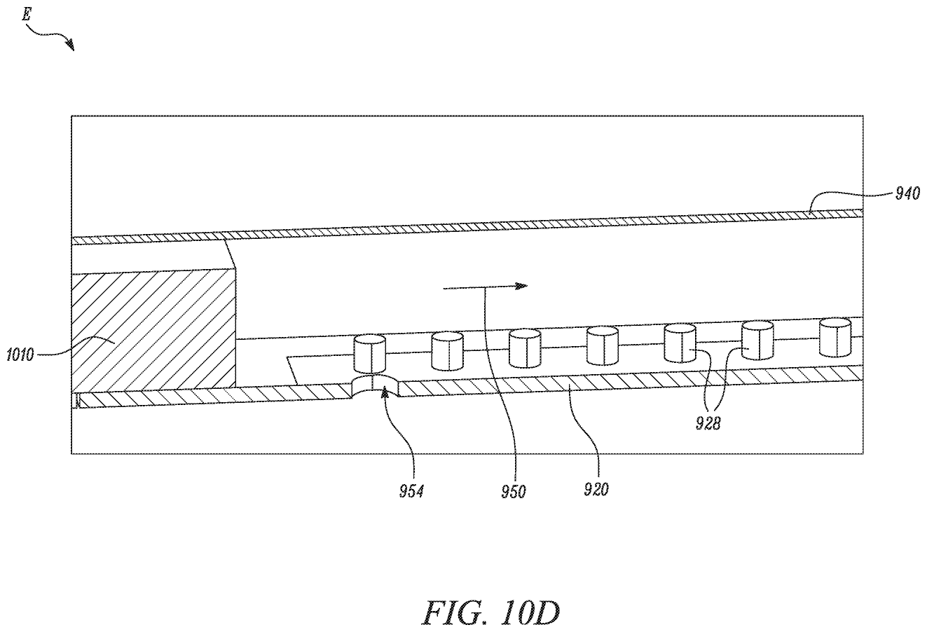

FIG. 10A illustrates a schematic exploded perspective view of a dressing device 1000 for the NPWT system 100 of FIG. 1, according to another embodiment of the present disclosure. FIG. 10B illustrates a schematic top view of the dressing device 1000, according to an embodiment of the present disclosure. FIG. 10C illustrates a magnified schematic exploded sectional side view of a portion D of the dressing device 1000 of FIG. 10B, according to an embodiment of the present disclosure. FIG. 10D illustrates a magnified schematic sectional perspective view of a portion E of the dressing device 1000 of FIG. 10B, according to an embodiment of the present disclosure.

The dressing device 1000 is functionally equivalent to the dressing device 900 shown in FIGS. 9A to 9E. Same parts are referred to herein by same numbers. However, referring to FIGS. 10A-10C, the dressing device 1000 further includes an intermediate manifold 1010 disposed between the manifold 920 and the top film 940. Specifically, the dressing device 1000 further includes the intermediate manifold 1010 disposed between the first portion 922 of the manifold 920 and the top film 940. In some embodiments, the intermediate manifold 1010 and the first portion 922 of the manifold 920 have the same shape and dimensions.

Referring now to FIGS. 10A and 10D, in some embodiments, the intermediate manifold 1010 includes a through-slot 1015 at least partially aligned with each of the manifold channel 926 and the top channel 942, such that the through-slot 1015 fluidly communicates the manifold channel 926 with the top channel 942. Thus, the top channel 942, the through-slot 1015, and the manifold channel 926 together define the pressure sensing pathway 950.

Referring to FIGS. 1 to 10D, overall, the dressing device 200, 400, 500, 600, 700, 800, 900, 1000 may be easy to use, may improve patient comfort, may reduce a possibility of infection at the wound site 12, may improve an efficacy and a portability of the NPWT system 100, and may effectively manage the wound exudate removed from the wound site 12.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

Spatially related terms, including but not limited to, “proximate,” “distal,” “lower,” “upper,” “beneath,” “below,” “above,” and “on top,” if used herein, are utilized for ease of description to describe spatial relationships of an element(s) to another. Such spatially related terms encompass different orientations of the device in use or operation in addition to the particular orientations depicted in the figures and described herein. For example, if an object depicted in the figures is turned over or flipped over, portions previously described as below, or beneath other elements would then be above or on top of those other elements.

As used herein, when an element, component, or layer for example is described as forming a “coincident interface” with, or being “on,” “connected to,” “coupled with,” “stacked on” or “in contact with” another element, component, or layer, it can be directly on, directly connected to, directly coupled with, directly stacked on, in direct contact with, or intervening elements, components or layers may be on, connected, coupled or in contact with the particular element, component, or layer, for example. When an element, component, or layer for example is referred to as being “directly on,” “directly connected to,” “directly coupled with,” or “directly in contact with” another element, there are no intervening elements, components or layers for example.

Various examples have been described. These and other examples are within the scope of the following claims.

1. A dressing device for a negative pressure wound therapy (NPWT) system, the dressing device comprising: a base film configured to couple to a wound site;
a manifold coupled to the base film, the manifold comprising a first portion configured to interface with the wound site and a second portion spaced apart from the wound site;
an absorbent spaced apart from the first portion of the manifold, wherein the absorbent is coupled to and extends along the second portion of the manifold, such that the absorbent is offset from the wound site; and
a top film disposed adjacent to the absorbent opposite to the manifold, such that the absorbent is disposed between the manifold and the top film.

2.-4. (canceled)

5. The dressing device of claim 1, wherein the first portion of the manifold comprises a plurality of perforations.

6. The dressing device of claim 5, wherein the first portion of the manifold comprises a plurality of projections extending opposite to the base film, the plurality of perforations in the first portion alternating with the plurality of projections in the first portion.

7. The dressing device of claim 1, wherein the second portion of the manifold comprises a plurality of projections extending opposite to the base film and at least partially engaging the absorbent.

8. The dressing device of claim 1, wherein the base film comprises a cutout at least partially aligned with the manifold.

9. The dressing device of claim 8, wherein a shape of the cutout is similar to a shape of the manifold.

10. The dressing device of claim 8, wherein the cutout is at least partially aligned with the first portion of the manifold, and wherein a shape of the cutout is similar to a shape of the first portion of the manifold.

11.-27. (canceled)

28. The dressing device of claim 1, wherein the second portion of the manifold extends from the first portion of the manifold along a longitudinal axis.

29. The dressing device of claim 28, wherein the first portion of the manifold comprises a first maximum width perpendicular to the longitudinal axis, wherein the second portion of the manifold comprises a second maximum width perpendicular to the longitudinal axis, and wherein the first maximum width is greater than the second maximum width by a factor of at least two.

30. The dressing device of claim 28, wherein the first portion of the manifold comprises a first length along the longitudinal axis, wherein the second portion of the manifold comprises a second length along the longitudinal axis, and wherein the second length is greater than or equal to the first length.

31. The dressing device of claim 1, wherein the top film is fluid permeable.

32. The dressing device of claim 1, wherein the y6g absorbent comprises a first major surface disposed adjacent to the top film and a second major surface opposite to the first major surface, and wherein the first major surface of the absorbent forms a pressure sensing region.

33.-39. (canceled)

40. A dressing device for a negative pressure wound therapy (NPWT) system, the dressing device comprising: a base film configured to couple to a wound site;
a manifold coupled to the base film, the manifold comprising a first portion configured to interface with the wound site and a second portion spaced apart from the wound site, wherein the manifold further comprises a manifold channel extending along the manifold and fluidly communicating with the wound site; and
a top film disposed adjacent to the manifold opposite to the base film, such that the manifold is disposed between the base film and the top film, wherein the top film comprises a top channel extending along the top film and aligned with the manifold channel, and wherein the manifold channel and the top channel together define a pressure sensing pathway therebetween.

41.-46. (canceled)

47. The dressing device of claim 40, further comprising a pad assembly coupled to the top film, wherein the pad assembly comprises a pressure sensing channel disposed in fluid communication with the pressure sensing pathway and configured to fluidly communicate with a pressure sensing device spaced apart from the top film.

48. The dressing device of claim 47, wherein the pad assembly further comprises a negative pressure channel fluidly isolated from the pressure sensing channel and disposed in fluid communication with the top film and configured to fluidly communicate with a negative pressure source, and wherein the manifold and the top film together form a negative pressure pathway fluidly communicating the wound site with the negative pressure channel.

49. The dressing device of claim 40, further comprising an intermediate manifold disposed between the manifold and the top film, wherein the intermediate manifold comprises a through-slot at least partially aligned with each of the manifold channel and the top channel, such that the through-slot fluidly communicates the manifold channel with the top channel.

50. The dressing device of claim 49, wherein the intermediate manifold and the first portion of the manifold have the same shape and dimensions.

51. The dressing device of claim 40, wherein the manifold channel extends at least partially along each of the first portion of the manifold and the second portion of the manifold.

52. The dressing device of claim 40, wherein the manifold channel extends along a longitudinal axis, wherein the first portion of the manifold comprises a first maximum width perpendicular to the longitudinal axis, wherein the second portion of the manifold comprises a second maximum width perpendicular to the longitudinal axis, and wherein the first maximum width is greater than the second maximum width by a factor of at least two.

53. The dressing device of claim 52, wherein the first portion of the manifold comprises a first length along the longitudinal axis, wherein the second portion of the manifold comprises a second length along the longitudinal axis, and wherein the second length is greater than or equal to the first length.

57. (canceled)
